(19) United States
(12) Patent Application Publication
(10) Pub. No.: US 2022/0071922 A1
(43) Pub. Date: Mar. 10, 2022

(54) DELIVERY OF HEALTH BENEFITS THROUGH COMPONENT FORMULATION

(71) Applicant: Clear Lake Research, LLC, Palo Alto, CA (US)

(72) Inventor: Douglas Thomas, Palo Alto, CA (US)

(73) Assignee: Clear Lake Research, LLC, Palo Alto, CA (US)

(21) Appl. No.: 17/419,242

(22) PCT Filed: Dec. 23, 2019
(86) PCT No.: PCT/US2019/068399

§ 371 Date: Jun. 28, 2021

Related U.S. Application Data
(60) Provisional application No. 62/785,180, filed on Dec. 26, 2018.

(51) Int. Cl. A61K 31/05; A61K 9/06

(52) U.S. Cl. CPC A61K 31/05; A61K 9/06

(57) ABSTRACT

Disclosed are cannabidiol (CBD) formulations with high CBD content for providing a variety of health benefits, and a system for the extraction and packaging thereof. Embodiments include a soothing formulation, an acne treatment formulation, and a sunburn treatment formulation. A method of treating discomfort susceptible to treatment with an anti-inflammatory substance delivered to a site of inflammation is also disclosed.

### BACKGROUND

Two primary endocannabinoid receptors have been identified: CB₁, first cloned in 1990; and CB₂, cloned in 1993. CB₁ receptors are found predominantly in the brain and nervous system, as well as in peripheral organs and tissues, and are the main molecular target of the endocannabinoid ligand (binding molecule), anandamide, as well as its mimetic phytocannabinoid, tetrahydrocannabinol (THC). One other main endocannabinoid is 2-arachidonoylglycerol (2-AG) which is active at both cannabinoid receptors, along with its own mimetic phytocannabinoid, cannabidiol (CBD). 2-AG and CBD are involved in the regulation of appetite, immune system functions, and pain management. “Cannabidiol: An Overview of Some Pharmacological Aspects,” by Mechoulam et al. (Journal of Clinical Pharmacology, Vol. 42, No. 51, pp. 11S-19S, November 2002) is hereby incorporated by reference herein.

### BRIEF DESCRIPTION OF THE DRAWINGS

FIG. 1 depicts a diagram of an example of a system for extraction and packaging of enriched extract.

### DETAILED DESCRIPTION

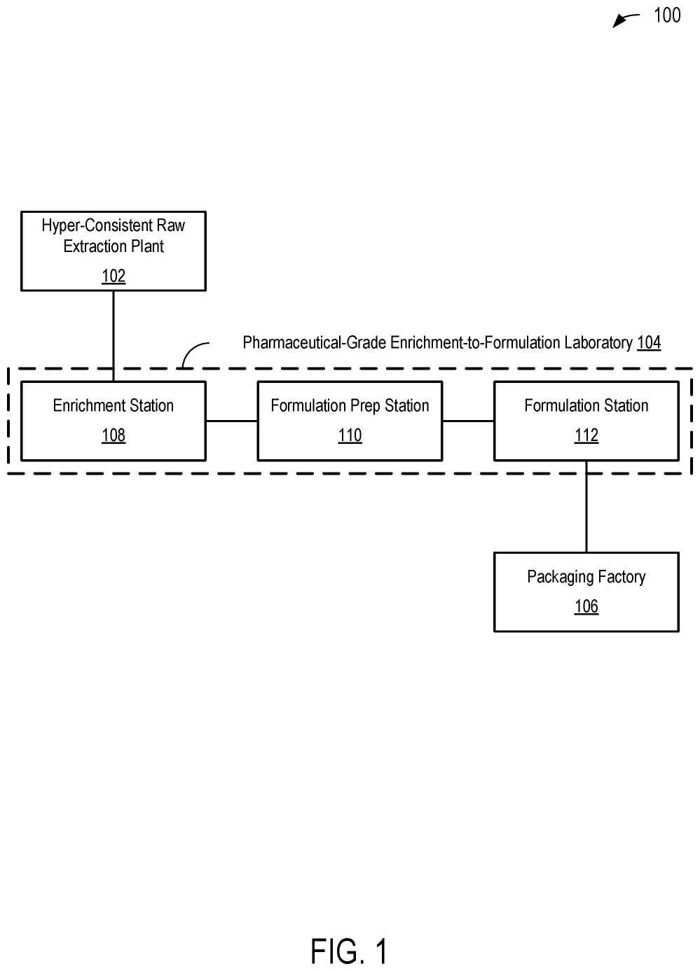

FIG. 1 depicts a diagram 100 of an example of a system for extraction and packaging of enriched extract. The diagram 100 includes a hyper-consistent raw extraction plant 102, a pharmaceutical-grade enrichment-to-formulation laboratory 104 coupled to the hyper-consistent raw extraction plant 102, and a packaging factory 106 coupled to the pharmaceutical-grade enrichment-to-formulation laboratory 104. In the diagram 100, the pharmaceutical-grade enrichment-to-formulation laboratory 104 includes an enrichment station 108, a formulation prep station 110, and a formulation station 112.

The hyper-consistent raw extraction plant 102 is intended to represent one or more devices (e.g., a facility) that receives a genetic strain of hemp bred for high cannabidiol (CBD) content, which is used to create a crude oil-soluble hemp extract with at least 80 mass percent CBD, or at least 90 mass percent CBD for high-end formulations, though we have achieved 99.98 mass percent CBD. Hyper-consistency is desirable to ensure predictability in the use of CBD in pharmacological applications. At below 80 mass percent CBD, hyper-consistency has not been achieved.

For pharmaceuticals or formulations that use pharmaceutical-grade techniques the raw material must always be hyper-consistent. As used in this paper, hyper-consistent is intended to mean “follows pharmaceutical standards.” Depending upon the jurisdiction, treatment products may be divided into cosmeceuticals, over-the-counter (OTC) pharmaceuticals, and prescription pharmaceuticals. Unlike cosmeceuticals and OTC pharmaceuticals, prescription pharmaceuticals must be prescribed by a licensed medical practitioner in accordance with medical guidelines due to the risk of adverse effects and contraindications. The precise distinction between cosmeceuticals, OTC pharmaceuticals, and prescription pharmaceuticals depends on the legal jurisdiction, but all components must be permitted by statute, such as the Federal Food, Drug, and Cosmetic Act (FFDCA) in the United States; generally recognized as safe (GRAS) by the relevant regulatory body, such as the United States Food and Drug Administration (FDA); and follow a relevant pharmaceutical process in accordance with current good manufacturing processes (cGMP). See, e.g., U.S. Code of Federal Regulation, Title 21, parts 210, 211, 212, 225, and 226, which are hereby incorporated by reference herein. Bioactives must be GRAS and Effective (GRASE), which means they will have undergone clinical research to determine dosage and a safety profile. Alternative medicines can include bioactives that have not yet been assessed and therefore are considered neither cosmeceuticals nor pharmaceuticals, but manufacturers of alternative medicine products can still follow pharmaceutical standards.

In an example of operation, the hyper-consistent raw extraction plant 102 includes one or more devices to macerate hyper-consistent hemp (e.g., via a macerator), add volatiles (e.g., via a nozzle), add carrier oil (e.g., via the same or another nozzle), remove the volatiles (e.g., via production-scale higher performance liquid chromatography (HPLC), gas chromatography, or distillation), and granulate (e.g., via a lyophilizer). Advantageously, granules have a low shipping weight and can be stored in bulk. Hyper-consistency facilitates the use of natural materials (e.g., hemp extract) in lieu of processed chemicals while ameliorating side effects, including increasing the probability of desirable effects that are inconsistently observed due to the presence of adulterants. While a “full spectrum” approach may be considered desirable in some products, consistency is necessary for pharmaceutical-grade products. Specifically, each component can work 1) for, 2) against, 3) with, or 4) n/a relative to each other component, and consistency enables control of the interplay between the various components. Moreover, it is well-understood that diluted solutions displace bioactive (or support) substance volume.

The pharmaceutical-grade enrichment-to-formulation laboratory 104 is intended to represent one or more devices (e.g., in a facility) that receive hyper-consistent crude oil soluble hemp extract from the hyper-consistent raw extraction plant 102. Where control is passed from one enterprise to another, it may be desirable to test received crude oil soluble hemp extract via mass spectrometry or HPLC and insist upon a specific compound profile to act like a pharmaceutical process in accordance with cGMP. It is assumed the crude oil soluble hemp extract used by the pharmaceutical-grade enrichment-to-formulation laboratory 104 is hyper-consistent in this paper. Because formulation can be up to ⅔ of the production cost of applicable products, it is important to make sure the components are of the desired quality, which in this example means the crude oil soluble hemp extract is hyper-consistent.

In a specific implementation, the enrichment station 108 purifies phytocannabinoid from the hyper-consistent crude oil soluble hemp extract. For example, HPLC can be used for fractionation. Fractionation is a separation process in which a certain quantity of a mixture (gas, solid, liquid, enzymes, suspension, or isotope) is divided during a phase transition, into a number of smaller quantities (fractions) in which the composition varies according to a gradient. Fractions are collected based on differences in a specific property of the individual components. A common trait in fractionations is the need to find an optimum between the amount of fractions collected and the desired purity in each fraction. Fractionation makes it possible to isolate more than two components in a mixture in a single run. This property sets it apart from other separation techniques.

In a specific implementation, the formulation prep station 110 transforms the enriched hyper-consistent crude oil soluble hemp extract and other components (ingredients) into a form suitable for formulation. For example, a lyophilizer, mentioned previously, can be used for granulation of components. An advantage of granulation is the component is dispersible, easy to measure, and easily formulable, but other forms (liquid being a common one) can be used.

In a specific implementation, the formulation station 112 exposes the components to one or more phases, including encapsulation, hot process, and cold process phases. Encapsulation, which can include solid phase encapsulation, can include both hot and cold phases, depending upon the components. The hot process can entail blending oils, dissolving salts, co-blending with an emulsifier, or the like. The cold process is used primarily when adding heat-sensitive components. Any of the processes can be used multiple times, though, of course, it may be undesirable to expose a heat-sensitive component to a subsequent hot process. Accordingly, the cold process is often the final process during which heat-sensitive components are blended in, e.g., liposomes.

The packaging factory 106 is intended to represent one or more devices (e.g., in a facility) that combine the components of a formulation from the pharmaceutical-grade enrichment-to-formulation laboratory 104 in packaging materials. Because phytocannabinoids interact with oxygen (giving CBD a shelf life of about six months), a nitrogen overlay with seal may be desirable (to increase shelf life). However, a nitrogen overlay with seal can shift the manufacturing costs from ⅓ packaging costs to around ½ packaging costs. Instead or in addition, an encapsulant can be used to increase shelf life. Interactions with other compounds can also impact shelf life, with preservatives actually increasing shelf life, so the formulation itself can also improve or degrade the shelf life of phytocannabinoids. In all instances, it is desirable to include an expiration date to ensure a consumer receives a product with consistent efficacy.

An example of a product (soothing cream) will now be discussed. The specific implementation described can be characterized as a vanishing soothing cream formulation with:
2% CBD
0.9% Menthol
1% Arnica Extract
1% Tea Tree Oil
1% Retinyl Palmitate
1% Tetrahexyldecyl Ascorbate

CBD has dual-activity in that it addresses both pain and inflammation. “Cannabidiol: From an Inactive Cannabinoid to a Drug with Wide Spectrum of Action,” by Zuardi (Brazilian Journal of Psychiatry, Vo. 30, No. 3, pp. 271-280, September 2008); “An Entourage Effect: Inactive Endogenous Fatty Acid Glycerol Esters Enhance 2-Arachidonoyl-Glycerol Cannabinoid Activity,” by Ben-Shabat et al. (European Journal of Pharmacology, Vol. 353, No. 1, pp. 23-31, July 1998); and “2-Arachidonoylglycerol (2-AG) Membrane Transport: History and Outlook,” by Hermann et al. (The AAPS Journal, Vol. 8, No. 2, pp. E409-E412, June 2006) are hereby incorporated by reference herein. Among other features, CBD binds to the CB₂ receptor on T-Cells, causing a reduction in inflammatory mediators, and binds to the transient receptor potential cation channel subfamily V member 1 (TrpV1), which is a pain receptor also known as the capsaicin receptor and the vanilloid receptor 1. Thus, CBD can be characterized as operating on the cell level to reduce both pain and inflammation.

Because CB₂ receptors have a low affinity for CBD, a relatively large amount of CBD should be used for efficacy. In a specific implementation, a soothing cream is 2% CBD. In alternatives, the soothing cream is as little as 0.5% CBD. Because CBD is a relatively expensive component, the ratio of CBD to other components is more prone to cost/efficacy trade-offs than other components of the formulation. The proportion could increase up to 5% for high-end CBD formulations. A greater proportion may also be desirable for the purpose of binding to theorized CBD3 receptors or other presently poorly-defined receptors with less affinity for CBD. If CBD is replaced with by endocannabinoid with (an estimated) 1,000 times the affinity of CBD, such as 2-Lineoleoyl-glycerol (3443-82-1) or 2-Palmitolyl-glycerol (23470-00-0), an alternative implementation of the soothing cream could have 0.0005% to 0.002% endocannabinoid, but the formulation would likely be much more expensive than CBD.

CBD has a log P of 7, which means CBD is extremely penetrating. (The partition coefficient, P, is defined as a particular ratio of the concentrations of a solute between the two solvents, a biphase of liquid phases, specifically for un-ionized solutes, and the logarithm of the ratio is thus log P.) Other endocannabinoids also typically have relatively high log P. It typically takes about 30 minutes for CBD, applied topically, to have a significant pain relief impact (though the onset of pain relief may be somewhat faster when applied to mucosa than it is when applied to skin and some endocannabinoids may have slightly different log P, which can impact the time).

For a more immediate impact, the CBD can be combined with menthol, which also binds to the TrpV1 receptor, but with an immediate response. Less than 1% menthol is necessary to avoid a product from acquiring an OTC designation; 1% or higher concentration of menthol is allowed in OTC medicines. Camphor can be used in lieu of (or in addition to) menthol but is not as effective and can cause a rash. Some products include both menthol and camphor to increase effectiveness without exceeding limits imposed on the components (e.g., to fall within OTC limits for both of the components). Wintergreen oil (methyl salicylate) can also be used, but overdose is possible (at least one death has been reported from its use in high concentrations), making it less safe as a component in a soothing lotion. In a specific implementation, a soothing cream is nearly 1% menthol (just below the limit that would cause the formulation to be governed by pharmaceutical laws and regulations), but anything over about 0.1% menthol would likely have some effect. Of course, if the formulation is for an OTC product, up to the regulated limits of menthol can be used, if desired.

Arnica, a member of the Asteraceae (sunflower) family, includes helenin, which is a mixture of two isomeric sesquiterpene lactones, alantolactone and isoalantolactone. Alantolactone, in vitro at least, exhibits anti-inflammatory effects by inhibiting chemokine production and STAT1 phosphorylation (and also appears to have anti-fungal, anti-microbial, STAT3 activation suppression characteristics). Helenin is a leukotriene, a family of eicosanoid inflammatory mediators produced in leukocytes by the oxidation of arachidonic acid (AA) and the essential fatty acid eicosapentaenoic acid (EPA) by the enzyme arachidonate 5-lipoxygenase. Although helenin can be characterized as operating at the enzyme (anti-inflammatory mediator) level, because helenin is log P 1.7, it is rarely delivered to the site of bioactivity (e.g., to treat inflammation) as a topical treatment. Thus, despite in vitro bioactivity, arnica in lotions is essentially bioinactive.

Tea tree oil (melaleuca oil) is used as a folk medicine to treat dandruff, acne, lice, herpes, insect bites, scabies, and skin fungal or bacterial infections, but the quality of the evidence for efficacy in these conditions is minimal. However, tea tree oil includes eucalyptol (1,8-cineol), which is a cyclic ether and monoterpenoid. Advantageously, eucalyptol is a penetration enhancer. As such, including, e.g., arnica in a formulation with eucalyptol turns the arnica into an actual bioactive by facilitating penetration through skin to a site of inflammation. Tea tree oil also has a preservative (antibacterial) effect in formulations and a fragrance.

Helenin, which operates at the enzyme level, eucalyptol, which enhances helenin penetration to a site of inflammation (rendering the helenin bioactive), and CBD, which operates at the cell level, when used in combination, enhance the anti-inflammatory efficacy of helenin alone, helenin combined with eucalyptol, and CBD alone. Moreover, the combination may also increase the shelf life of CBD when the eucalyptol acts as a preservative of the CBD. In a specific implementation, a soothing cream is 1% arnica extract and 1% tea tree oil, though less is needed for helenin or eucalyptol, respectively, which is a benefit of greater purity. In alternatives, tea tree oil ranges from 0.5% to 4% and arnica ranges from 0.5% to 7%. At 0.5%, tea tree oil is a poor penetration enhancer and has minimal fragrance, but is still effective as a preservative, while at 4%, tea tree oil is an exceptional penetration enhancer and antibacterial. Arnica ranges provide less (0.5%) or more (7%) anti-inflammatory effect.

An antimicrobial preservative system is standard practice for bioactive treatment modalities, but the mass amount tends to be quite low (typically as low as 0.01%, though up to 1% is possible). In a specific implementation, an additional preservative system is present in the formulation.

A soothing cream can include a muscle relaxant as a component. In a specific implementation, this is CBD, which is known to act as a muscle relaxant. However, in an alternative, a phytocannabinoid can be augmented with a primary (or secondary) muscle relaxant component.

A soothing cream can include an endocannabinoid mimetic, such as a synthetic cannabinoid, as a component. Examples of endocannabinoid mimetics are 18-Hydroxypalmitic acid (Juniperic acid), AM404 (18718-77-6), and guineesine. In an alternative that includes, e.g., Juniperic acid and CBD, the Juniperic acid enables the CBD to linger for longer. Advantageously, because the “side effects” are reduced pain and inflammation, the composition would be safe as a topical treatment.

Emulsifiers typically have a polar (hydrophilic) and a non-polar (lipophilic) part and are used to increase kinetic stability. Emulsifiers that are more soluble in water are generally used to form O/W solutions and emulsifiers that are more soluble in oil are generally used to form W/O emulsions. An oil-in-water (O/W) solution is creamy and is generally better for hydration than a water-in-oil (W/O) solution (and may be considered to have a more desirable touch and feel). Accordingly, in a specific implementation, a soothing cream includes an emulsifier. As used in this paper, a cream is an O/W emulsion or aqueous microcrystalline dispersion of long-chain fatty acids or alcohols that are water washable. Creams may include more than 20% water or volatile components and less than 50% hydrocarbons, waxes, or polyols as vehicles. Other topical medication forms include ointment (20% water), gel, paste, powder, and liniment. A lotion is a low-viscosity topical preparation intended for application to unbroken skin. In a specific implementation, a soothing cream is about 30% water, which can be characterized as a “day cream;” night creams tend toward ointment-level ratios (e.g., 20% water) and a soothing lotion could have as high as 50% water.

A soothing cream can include an antioxidant. Tetrahexyldecyl ascorbate, a lipophilic form of vitamin C, an antioxidant. Because tetrahexyldecyl ascorbate is lipophilic, the vitamin C is more readily absorbed in the skin. When absorbed in the skin, vitamin C is known to effect production of connective tissue, resulting in new and strengthened skin. In a specific implementation, a soothing cream is 1% tetrahexyldecyl ascorbate. At 1%, tetrahexyldecyl ascorbate acts to stabilize fatty acids that are inherently moderately oxidation-sensitive bioactives. In alternatives, tetrahexyldecyl ascorbate ranges from 0.1% to 5%, where at the lower range, tetrahexyldecyl ascorbate acts as a cream stabilizer while at the upper range tetrahexyldecyl ascorbate is dual-active as a cream stabilizer and oxidation-sensitive bioactive stabilizer.

We believe inflammation can be further reduced by using an additional antiinflammation component, retinyl palmitate. After absorption into the skin, retinyl palmitate is converted to retinaldehyde, and ultimately to retinoic acid (the active form of vitamin A present in Retin-A). A penetration enhancer, such as eucalyptol, can improve the efficacy of retinyl palmitate for this purpose. Retinoic acid is an antioxidant. In a specific implementation, a soothing cream is 1% retinyl palmitate. Further, retinyl palmitate can act to stabilize CBD against photonic oxidation and the resulting discoloration. However, it should be noted retinyl palmitate at 1% ratio may result in as little as 0.05% retinaldehyde penetrating the skin; as little as 0.05% retinaldehyde could achieve similar efficacy because almost all can be absorbed through the skin. In alternatives, retinyl palmitate ranges from 0.1% to 5%, where at the lower range retinyl palmitate provides protection from the sun, but is less effective as an antioxidant, and at the upper range retinyl palmitate can provide SPF-level sun protection in addition to acting as a secondary antioxidant. The higher range is also used when penetration is relatively poor or when a greater amount of retinoic acid is needed for some purpose.

An acne treatment cream will now be discussed. The specific implementation described can be characterized as a cream formulation with:
1% CBD
1% Tea Tree Oil
1% Zinc Gluconate
1% Short Chain HA
0.05% Retinaldehyde
6% Glycolic Acid
1% Tetrahexyldecyl Ascorbate

Known acne treatment regimens focus on one to four of the four pillars of acne treatment: reducing sebum (used as fuel by bacteria) production, retarding bacterial growth, antiinflammation, and alleviation of hyperkeratosis. As will be discussed below, CBD addresses three new pillars of acne treatment. The first (0th pillar) is cellular quiescence; CBD binds to an expected CBD3 receptor, which shuts down sebum production by causing the cells to quiet. CBD is an anti-inflammatory, addressing the third pillar, albeit via a new modality. CBD reduces pain associated with both inflammation and comedone formation (a new fourth pillar). CBD promotes re-healing by decreasing inflammation in coordination with glycolic acid, which exfoliates and promotes skin growth. (The second and fourth pillars are addressed by other components of the formulations described below.)

In addition to its other effects, CBD exerts sebostatic effects on human sebocytes. Advantageously, CBD can not only bind to recently discovered sebocyte receptors, which should shut down sebum production via cellular quiescence (a new “0th pillar”) and act as a topical antiinflammation treatment (the third pillar), but also co-bind to endocannabinoid system (ECS) receptors, which should reduce pain (a new fourth pillar), and promote tissue re-healing (a new fifth pillar). “Cannabidiol Exerts Sebostatic and Anti-Inflammatory Effects on Human Sebocytes,” by Olah et al. (Journal of Clinical Investigation, Vol. 124, No. 9, pp. 3713-3724, September 2014) and “An Endogenous Cannabinoid (2-AG) is Neuroprotective After Brain Injury,” by Panikashvili et al. (Nature, Vol. 413, pp. 527-531, October 2001) are hereby incorporated by reference herein. In a specific implementation, an acne treatment cream is 1% CBD. In alternatives, the soothing cream is as little as 0.5% CBD or as much as 5%.

Retinoic acid, a metabolite of retinal (vitamin A), operates at the molecular level where it binds to the retinoid X receptor (RXR), causing a reduction in sebum production. This can be advantageous in a soothing cream, but has additional advantages when used as a skin product designed to reduce sebum production. Because retinoic acid binds to RXR, it addresses the first of the four pillars of acne treatment. Retinoic acid also acts at the enzyme level to decrease hyperkeratosis (the fourth pillar).

Retinaldehyde is converted in the body to retinoic acid. While retinal could be used and is in some acne products, retinal is less stable and requires a more complicated conversion to retinoic acid; accordingly, the prior art is inferior. In a specific implementation, an acne treatment cream is 0.05% retinaldehyde. 0.05% is a desired therapeutic level. In alternatives, retinaldehyde ranges from 0.01% to 0.1%, where at the lower range efficiency across the skin is lower, but the upper range is limited to avoid reaching a level of toxicity, which is not a risk at about 0.1%.

Glycolic acid finds applications in skin care products, most often as a chemical peel performed by a dermatologist in concentrations of 20 to 70% or at-home kits in lower concentrations between 10 and 20%. Once applied, glycolic acid reacts with the upper layer of the epidermis as a keratolytic, weakening the binding properties of the lipids that hold dead skin cells together. This allows the stratum corneum to be exfoliated, exposing live skin cells and smoothing skin. Highly purified grades of glycolic acid are commercially available for personal care applications. In a specific implementation, an acne treatment cream is 6% glycolic acid. Advantageously, at 6% glycolic acid, the composition is less damaging to clothing than at 10% or higher and the composition can be used as a leave-on cream. In alternatives, glycolic acid ranges from 2% to 20%. In alternatives, some other keratolytic, such as salicylic acid, urea, or sulfur, could replace the glycolic acid.

Ralga has a defined ratio between glycolic acid (6%) and retinaldehyde (0.05%). Ralga has known beneficial rejuvenative effects with good tolerability. Thus, adjusting the ratio of glycolic acid may make it desirable to adjust the ratio of retinaldehyde, as well.

Hyaluronic acid (HA) is a natural, ubiquitous stable polymer filler not typically used as a bioactive in topicals because it does not penetrate skin. HA will elicit a beta-defensin 2 production response in human skin because it cross-reacts with a bacterial signature, which retards bacterial growth (the second pillar). Low molecular weight (less than 200 kDa) hyaluronic acid (HA) has some penetrative capabilities and at around 150 kDa, HA would be able to penetrate skin with low penetration efficiency. Short-chain HA is defined as low molecular weight HA of 1-50 kDa. Advantageously, short chain HA has high penetration efficiency. In a specific implementation, an acne treatment cream is 1% short chain (20 kDa) HA. In alternatives, short chain HA ranges from 0.05% to 5%. The shorter the chain, the less HA is needed for a therapeutic result, so at the lower range, short chain (1 kDa) HA can be used and at the upper range, short chain (50 kDa) HA can be used.

As mentioned previously, tea tree oil also has a preservative effect in formulations and a fragrance. Because topical acne treatments are generally applied to abraded skin, there is limited need for a penetration enhancer, which tea tree oil is, but tea tree oil can still act as an antibacterial (the third pillar). In a specific implementation, an acne treatment cream is 1% tea tree oil. In alternatives, tea tree oil ranges from 0.5% to 4%.

The new fifth pillar can be addressed with tissue re-healing components. LL-37 (or CAP-18 for cathelicidin antimicrobial peptide, 18 kDa) is a gene encoding for the only member of the human cathelicidin family. Cathelicidin-related antimicrobial peptides are a family of polypeptides found in lysosomes of macrophages and polymorphonuclear leukocytes (PMNs), and keratinocytes. Cathelicidins serve a critical role in mammalian innate immune defense which exhibits broad-spectrum microbicidal activity against bacteria, fungi, and viruses. SAAP-148 (a synthetic antimicrobial and antibiofilm peptide) is a modified version of LL-37 that has enhanced antimicrobial activities compared to LL-37. In particular, SAAP-148 is more efficient in killing bacteria under physiological conditions. Zinc and calcium have different direct effects that facilitate re-healing (and calcium also improves zinc uptake).

Zinc gluconate (or zinc sulfate, zinc chloride, zinc acetate, or the like) can be applied to skin and the zinc acts at an enzyme level to bolster defensin production. It may be desirable to include more than the effective dosage because the dead skin layer may degrade zinc uptake. Zinc gluconate can penetrate abraded skin, but an enhancer may otherwise be necessitated for optimal efficacy. In a specific implementation, an acne treatment cream is 1% zinc gluconate. In alternatives, the acne cream is less than 1% (as low as 0.1%) or more than 1% (as high as 2%). At the lower range, other enablers may be used in the formulation to increase uptake. At the higher range, high water content formulations may need a higher ratio of zinc gluconate to retain efficacy.

Calcium improves barrier function. Also, calcium chloride can be applied to the skin to improve the uptake of zinc, further increasing defensin production. Accordingly, an acne cream with zinc may also include calcium chloride. In a specific implementation, an acne treatment cream is 1% calcium chloride. In an alternative, the acne treatment cream is less than 1% (as low as 0.1%) or more than 1% (as high as 2%). At the lower range, other enablers may be used in the formulation to increase uptake. At the higher range, high water content formulations may need a higher ratio of calcium chloride to retain efficacy.

With both zinc and calcium, penetration efficiency is low. For this reason, it is generally desirable to include a bit more than what may be desired at the site of bioactivity. The excess remains (harmless) on the skin.

Trans epidermal water loss (TEWL) is higher with abraded skin. Humectants are common ingredients in cosmetic and personal care products and can include, e.g., propylene glycol, aloe vera, alpha hydroxy acids, honey, molasses, sugar alcohols, to name several. In the specific implementation provided above, an acne treatment cream does not include aloe vera, but in alternatives, aloe vera ranges from 1% to 10%. Oilier skin may react better to lower ratios, against invasive bacterial infection. Vitamin D up-regulates genetic expression of cathelicidin, while drier skin might react better to higher ratios. At some point, the consistency of the cream will become more like a gel; an acne-treating gel might have about 60% aloe vera. It may be noted humectants generally replace water in formulations, as opposed to bioactives. Also, humectants are difficult to incorporate into a vanishing cream, such as the soothing cream formulation describe above.

As mentioned previously, tetrahexyldecyl ascorbate is an antioxidant. In a specific implementation, an acne treatment cream is 1% tetrahexyldecyl ascorbate. In alternatives, tetrahexyldecyl ascorbate ranges from 0.1% to 5%, where at the lower range, tetrahexyldecyl ascorbate acts as a cream stabilizer while at the upper range tetrahexyldecyl ascorbate is dualactive as a cream stabilizer and oxidation-sensitive bioactive stabilizer.

A sunburn treatment cream will now be discussed. The specific implementation described can be characterized as a leave-on cream formulation with:
2% CBD
0.9% Menthol
1% Avena Sativa
1% Retinyl Palmitate
1% Short Chain HA
10% Aloe Vera

The components of the sunburn cream for the specific implementation provided above were describe previously, except for Avena sativa (oat), which is an anti-inflammatory. Avenanthramides are a group of phenolic alkaloids found mainly in oats, but also present in white cabbage butterfly eggs, and in fungus-infected carnations. A number of studies demonstrate that these natural products have anti-inflammatory, antioxidant, anti-itch, anti-irritant, and antiatherogenic activities. Avena sativa provides its anti-inflammatory and other benefits on the enzyme level. In a specific implementation, a sunburn treatment cream is 1% Avena sativa. In alternatives, the Avena sativa ranges from 0.1% to 3%. In general, the ratio will depend upon log P.

As was previously indicated, retinyl palmitate can provide SPF-level sun protection at higher ratios. Accordingly, an alternative sunburn treatment that also acts as a sunscreen includes 5% retinyl palmitate. Advantageously, such an implementation could provide sunscreen protection that did not cause pain when applied to already-burned skin.

In a specific implementation, sunburn treatment cream is about 5% aloe vera. A humectant is likely desirable in sunburn treatment creams and, as was mentioned previously, increasing the ratio higher will result in a formulation becoming a gel, rather than a cream.

Advantageously, CBD can be combined with a sebocyte-addressing component and enzyme inhibitors and, as has been explained above, the components can be all-natural, or even organic, with the proper certification.

These and other examples provided in this paper are intended to illustrate but not necessarily to limit the described implementation. As used herein, the term “implementation” means an implementation that serves to illustrate by way of example but not limitation. The techniques described in the preceding text and FIGURES can be mixed and matched as circumstances demand to produce alternative implementations.

1. A composition of matter comprising:
0.5% to 5% cannabidiol (CBD);
less than 1% menthol;
no more than 7% anti-inflammatory mediator, wherein the anti-inflammatory mediator operates at the enzyme level but is essentially bioinactive on human skin unless combined with a penetration enhancer;
no more than 4% of the penetration enhancer;
0.1% to 5% antioxidant.

2. The composition of matter of claim 1 comprising 1% to 3% CBD.

3. The composition of matter of claim 1, wherein the anti-inflammatory mediator includes 0.5% to 7% Arnica sp. extract.

4. The composition of matter of claim 1, wherein the anti-inflammatory mediator includes no more than 3% arnica extract.

5. The composition of matter of claim 1, wherein the penetration enhancer is 0.5% to 4% tea tree oil.

6. The composition of matter of claim 1, wherein the penetration enhancer is no more than 3% tea tree oil.

7. The composition of matter of claim 1 comprising no more than 1% retinyl palmitate.

8. The composition of matter of claim 1 comprising no less than 1% retinyl palmitate.

9. The composition of matter of claim 1 wherein the antioxidant includes no more than 1% tetrahexyldecyl ascorbate.

10. The composition of matter of claim 1 wherein the antioxidant includes no less than 1% tetrahexyldecyl ascorbate.

11. The composition of matter of claim 1 comprising 0.1% to 5% retinyl palmitate.

12. The composition of matter of claim 1 comprising more than 20% water or volatile components.

13. The composition of matter of claim 1 comprising less than 50% hydrocarbons, waxes, or polyols.

14. The composition of matter of claim 1 comprising less than 4% camphor.

15. The composition of matter of claim 1, wherein the CBD is derived from a hemp extract having between 80-99.98 mass percent CBD.

16. The composition of matter of claim 1, wherein the CBD is derived from a hemp extract from which phytocannabinoid has been purified by fractionating the hemp extract using high performance liquid chromatography (HPLC), CO₂ Extraction, Olive Oil Extraction, Dry Ice, or Solvent Extraction methods.

17. The composition of matter of claim 1, wherein the composition of matter is exposed to one or more of encapsulation, a hot process phase, and a cold process phase.

18. The composition of matter of claim 1, wherein the composition of matter is packaged with a nitrogen overlay with a seal.

19. A composition of matter comprising:
0.0005% to 0.002% endocannabinoid;
less than 1% menthol;
no more than 7% anti-inflammatory mediator, wherein the anti-inflammatory mediator operates at the enzyme level but is essentially bioinactive on human skin unless combined with a penetration enhancer;
no more than 4% of the penetration enhancer;
0.1% to 5% antioxidant.

20. A method comprising:
a method of treating discomfort susceptible to treatment with an anti-inflammatory substance delivered to a site of inflammation, said method comprising administering topically to a patient, an effective amount of a composition applying a soothing cream comprising 0.5% to 5% cannabidiol (CBD); less than 1% menthol; no more than 7% anti-inflammatory mediator, wherein the anti-inflammatory mediator operates at the enzyme level but is essentially bioinactive on human skin unless combined with a penetration enhancer; no more than 4% of the penetration enhancer; and 0.1% to 5% antioxidant.
